(19) United States
(12) Patent Application Publication
(10) Pub. No.: US 2015/0309303 A1
(43) Pub. Date: Oct. 29, 2015

(54) MAGNIFICATION LOUPE WITH ENERGY-HARVESTING SYSTEM

(71) Applicant: ORANGEDENTAL GMBH & CO. KG, Biberach (DE)

(72) Inventors: Ludwig LAXHUBER, Herrsching (DE); Stefan KALTENBACH, Rebstein (CH)

(21) Appl. No.: 14/443,966

(22) PCT Filed: Nov. 12, 2013
(86) PCT No.: PCT/EP2013/073549

§ 371 Date: May 19, 2015

(30) Foreign Application Priority Data
Nov. 19, 2012 (EP) 12193265.1

(51) Int. Cl. G02B 25/02; G02C 11/04; A61B 1/24; F21L 4/08; H02J 7/02; G02B 25/00; F21V 33/00

(52) U.S. Cl. CPC G02B 25/02; G02B 25/004; G02C 11/04; F21V 33/0068; F21L 4/08; H02J 7/025; A61B 1/24; F21L 2001/00

(57) ABSTRACT

The present invention relates to optical instruments, in particular to magnification loupes, such as those worn by dentists and surgeons. The invention features magnification loupes that are equipped with a light source, an energy harvesting component and an energy storage component. The invention further relates to magnification loupes comprising a light source and a polymer battery as energy storage component. In addition, the invention is directed to magnification loupes comprising a first light source emitting a light beam and a sensor structured and configured to detect a reflection of this light beam. If such a reflection is detected, a second light source mounted on the magnification loupe is turned on or off. The invention further concerns a ring made from or coated with a light-reflecting material. The invention also relates to a battery charging station on which the batteries present in the magnification loupes can be recharged.

The present invention relates to optical instruments, in particular to magnification loupes, such as those worn by dentists and surgeons.

The invention features magnification loupes that are equipped with a light source, an energy harvesting component and an energy storage component. The invention further relates to magnification loupes comprising a light source and a polymer battery as energy storage component. In addition, the invention is directed to magnification loupes comprising a first light source emitting a light beam and a sensor structured and configured to detect a reflection of this light beam. If such a reflection is detected, a second light source mounted on the magnification loupe is turned on or off. The invention further concerns a ring made from or coated with a light-reflecting material. The invention also relates to a battery charging station on which the batteries present in the magnification loupes can be recharged.

### Background and Technical Problems Underlying the Present Invention

Magnification loupes used by dentists or surgeons typically comprise a spectacle frame with two spectacle lenses and two magnifying optical systems (one for each eye) that are fixed to the spectacle lenses. In addition, magnification loupes often comprise a light source so that the operating field is illuminated in surgical procedures or the patient's mouth is illuminated in dental examinations. In general, this light source consumes significant amounts of energy so that a (rechargeable) battery providing power to the light source is depleted in short time-periods and has to be replaced (or recharged) very often.

Accordingly, there was a need in the prior art for magnification loupes with light sources that provide the required illumination for extended time-periods so that batteries have to be replaced or recharged less often.

The present inventors constructed novel magnification loupes that address this need. The novel magnification loupes are described in the appended claims and in the following description.

The above overview does not necessarily describe all problems solved by the present invention.

### SUMMARY OF THE INVENTION

In a first aspect the present invention relates to a magnification loupe (1), comprising: (a) a light source (60) structured and configured to emit light in the visible spectrum, wherein the direction of the emitted light is parallel to the viewing direction of a person wearing the magnification loupe (1); (b) a spectacle frame (10); (c) a magnifying optical system (40); (d) an energy harvesting component (80) that converts acoustic energy or light energy to electric energy, thereby generating electric energy; and (e) an energy storage component (90) that is structured and configured to store electric energy received from the energy harvesting component (80) and that is further structured and configured to provide electric energy to the light source (60).

In a second aspect the present invention relates to a magnification loupe (1), comprising: (a) a light source (60) structured and configured to emit light in the visible spectrum, wherein the direction of the emitted light is parallel to the viewing direction of a person wearing the magnification loupe (1); (b) a spectacle frame (10), wherein said spectacle frame comprises an energy storage component (90), said energy storage component (90) being a polymer battery (92) that is structured and configured to provide electric energy to the light source (60); and (c) a magnifying optical system (40).

In a third aspect the present invention relates to a magnification loupe (1), comprising: (a) a light source (60) structured and configured to emit light in the visible spectrum, wherein the direction of the emitted light is parallel to the viewing direction of a person wearing the magnification loupe (1); (b) a spectacle frame (10); (c) a magnifying optical system (40); (d) a light source (100) structured and configured to emit an infrared (IR) light beam, wherein said IR light beam is parallel to the viewing direction of a person wearing the magnification loupe; (e) a sensor (110) structured and configured to detect whether the IR light beam generated by the light source (100) is reflected in the direction of the magnification loupe (1); said sensor (110) being structured and configured to activate said light source (60) when the following two conditions are fulfilled: (i) a reflection of the IR light beam is detected by the sensor (110) and (ii) the light source (60) is off, said sensor (110) further being structured and configured to deactivate said light source (60) when the following two conditions are fulfilled: (iii) a reflection of the IR light beam is detected by the sensor (110) and (iv) the light source (60) is on; and (f) an energy storage component (90) that is structured and configured to provide electric energy to the light source (60) and that is further structured and configured to provide electric energy to the light source (100).

In a fourth aspect the present invention relates to a ring (200) having dimensions that allow the ring to be placed around the opened mouth of a human being, wherein said ring reflects infrared light.

In a fifth aspect the present invention relates to a kit-of-parts comprising the magnification loupe (1) according to the third aspect and at least one ring (200) according to the fourth aspect.

In a sixth aspect the present invention relates to a battery charging station (300) that is formed so that the magnification loupe (1) according to any one of the first, second or third aspect can be placed on said battery charging station (300), wherein said battery charging station (300) is structured and configured to recharge the energy storage component (90) of the magnification loupe (1) by inductive charging when the magnification loupe (1) is placed on said battery charging station (300).

In a seventh aspect the present invention relates to a kit-of-parts comprising: at least one magnification loupe (1) according to any one of the first, second or third aspect; and a battery charging station (300) according to the sixth aspect.

This summary of the invention does not necessarily describe all features of the present invention. Other embodiments will become apparent from a review of the ensuing detailed description.

### BRIEF DESCRIPTION OF THE DRAWINGS

FIG. 1 shows a top plan view of a magnification loupe according to one embodiment of the present invention. The three arrows with the solid lines signify light beams emanating from light source (60). The viewing direction of the person wearing the magnification loupe is towards the bottom of the figure.

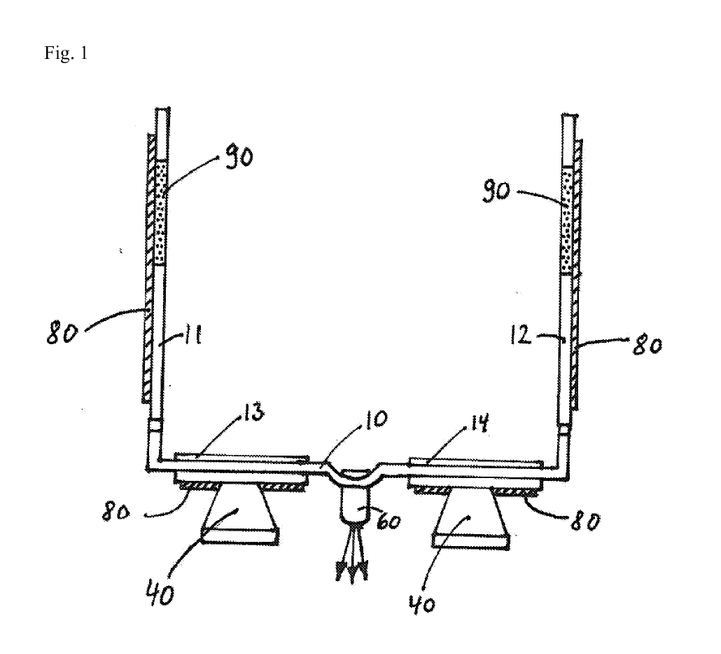

FIG. 2 shows a top plan view of a magnification loupe according to another embodiment of the present invention. The three arrows with the solid lines signify light beams emanating from light source (60). The viewing direction of the person wearing the magnification loupe is towards the bottom of the figure.

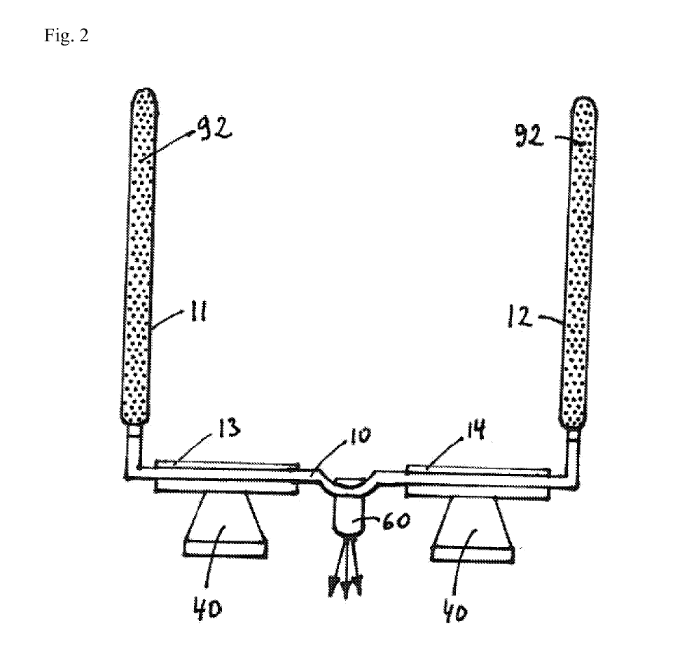

FIG. 3 shows a front view of a magnification loupe according to another embodiment of the present invention.

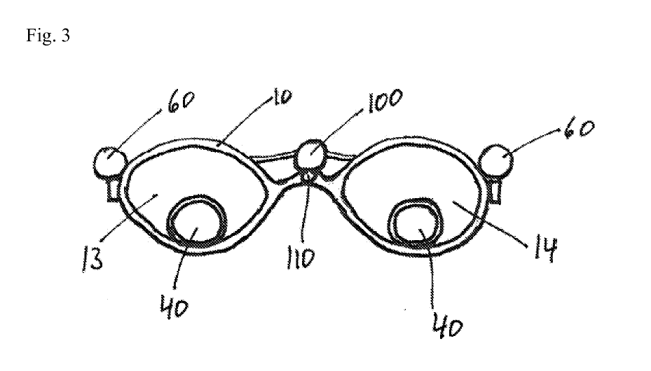

### DETAILED DESCRIPTION OF THE INVENTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art to which this invention belongs.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step.

Several documents (for example: patents, patent applications, scientific publications, manufacturer's specifications, instructions, etc.) are cited throughout the text of this specification. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

The term “about” when used in connection with a numerical value is meant to encompass numerical values within a range having a lower limit that is 5% smaller than the indicated numerical value and having an upper limit that is 5% larger than the indicated numerical value.

In the context of the present application, the term “user” refers to a person wearing the magnification loupe of the invention.

### Embodiments of the Invention

The present invention will now be further described. In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous, unless clearly indicated to the contrary.

In a first aspect the present invention is directed to a magnification loupe (1), comprising:

(a) a light source (60) structured and configured to emit light in the visible spectrum, wherein the direction of the emitted light is parallel to the viewing direction of a person wearing the magnification loupe (1);
(b) a spectacle frame (10);
(c) a magnifying optical system (40);
(d) an energy harvesting component (80) that converts acoustic energy or light energy to electric energy, thereby generating electric energy; and
(e) an energy storage component (90) that is structured and configured to store electric energy received from the energy harvesting component (80) and that is further structured and configured to provide electric energy to the light source (60).

The spectacle frame (10) typically comprises a right temple (11) and a left temple (12). The magnification loupe (1) of the first aspect may additionally comprise a right spectacle lens (13) and a left spectacle lens (14). The spectacle lenses (13, 14) need not be corrective lenses; they may be simple glass panes. However, for users that need corrective lenses, it is also possible to use such corrective lenses as the right and left spectacle lenses (13, 14) of the magnification loupe (1). The magnifying optical system (40) can be fixed to the right spectacle lens (13) and to the left spectacle lens (14).

In one embodiment of the first aspect, said energy harvesting component (80) converts acoustic energy to electric energy and is a piezoelectric element (81). In another embodiment of the first aspect, said energy harvesting component (80) converts light energy to electric energy and said energy harvesting component (80) is a photovoltaic cell (82). The magnification loupe (1) of the present invention may also comprise both an energy harvesting component (80) in the form of a piezoelectric element (81) and an energy harvesting component (80) in the form of a photovoltaic cell (82).

In one embodiment, the piezoelectric element (81) is located on the right spectacle lens (13) and/or on the left spectacle lens (14). In this embodiment, it is advantageous to use piezoelectric elements (81) that are transparent or at least partially transparent. The piezoelectric element (81) may cover a part of a spectacle lens (13, 14) or it may cover the complete front surface of a spectacle lens (13, 14), with the exception of the surface covered by the magnifying optical system (40). In another embodiment, the piezoelectric element (81) is located on at least one of the temples (11, 12) of the spectacle frame (10). In a further embodiment, the piezoelectric element (81) is located both on the spectacle lenses (13, 14) and on the temples (11, 12) of the spectacle frame (10).

In one embodiment, the photovoltaic cell (82) is located on the right spectacle lens (13) and/or on the left spectacle lens (14). In this embodiment, it is advantageous to use photovoltaic cells (82) that are transparent or at least partially transparent. The photovoltaic cell (82) may cover a part of a spectacle lens (13, 14) or it may cover the complete front surface of a spectacle lens (13, 14), with the exception of the surface covered by the magnifying optical system (40). In another embodiment, the photovoltaic cell (82) is located on at least one of the temples (11, 12) of the spectacle frame (10). In a further embodiment, the photovoltaic cell (82) is located both on the spectacle lenses (13, 14) and on the temples (11, 12) of the spectacle frame (10).

Also considered within the present invention are magnification loupes (1) in which the spectacle lenses (13, 14) are covered with photovoltaic cells (82) and the temples (11, 12) are covered with piezoelectric elements (81); or vice versa.

In preferred embodiments of the first aspect, the energy storage component (90) is a rechargeable battery (91).

The energy harvesting component (80) (e.g. a piezoelectric element (81) or a photovoltaic cell (82)) helps recharging the energy storage component (90) during use so that the user can wear the magnification loupe (1) for an extended period of time before the magnification loupe (1) has to be connected to a power supply, e.g. to a battery charging station (300).

In a second aspect the present invention is directed to a magnification loupe (1), comprising:

(a) a light source (60) structured and configured to emit light in the visible spectrum, wherein the direction of the emitted light is parallel to the viewing direction of a person wearing the magnification loupe (1);
(b) a spectacle frame (10), wherein said spectacle frame comprises an energy storage component (90), said energy storage component (90) being a polymer battery (92) that is structured and configured to provide electric energy to the light source (60); and
(c) a magnifying optical system (40).

The spectacle frame (10) typically comprises a right temple (11) and a left temple (12). The magnification loupe (1) of the second aspect may additionally comprise a right spectacle lens (13) and a left spectacle lens (14). The spectacle lenses (13, 14) need not be corrective lenses; they may be simple glass panes. However, for users that need corrective lenses, it is also possible to use such corrective lenses as the right and left spectacle lenses (13, 14) of the magnification loupe (1). The magnifying optical system (40) can be fixed to the right spectacle lens (13) and to the left spectacle lens (14).

In preferred embodiments of the second aspect, at least one of said temples (11, 12) is made from a polymer battery (92).

In preferred embodiments of the second aspect, the polymer battery (92) is a lithium ion polymer battery.

In particularly preferred embodiments of the second aspect, the polymer battery (92) is rechargeable.

One advantage of polymer batteries, especially lithium ion polymer batteries, is that these batteries can be made in almost any shape. While batteries used in magnification loupes of the prior art typically are standard size batteries that are inserted into a small compartment located in one temple of the spectacle frame, polymer batteries can be formed in such a manner that a complete temple (11, 12) of the spectacle frame (10) (or at least a large portion of the temple (11, 12)) is made from a polymer battery (92). This design leads to a significant increase in the volume of the battery that powers the magnification loupe (1), and accordingly, to a significant increase in the time-period in which sufficient power is available for light source (60). As a consequence, the user can wear the magnification loupe (1) for an extended period of time before the magnification loupe (1) has to be connected to a power supply, e.g. to a battery charging station, such as battery charging station (300). It should also be noted that another advantage of polymer batteries (92), especially lithium ion polymer batteries, is their low weight. Thus, the magnification loupe (1) according to the second aspect is not heavier than magnification loupes of the prior art.

It is also considered within the present invention to combine the advantages of the first aspect and the second aspect. Thus, the energy storage component (90) of the first aspect can also be a polymer battery (92), such as a lithium ion polymer battery.

In a third aspect the present invention is directed to a magnification loupe (1), comprising

(a) a light source (60) structured and configured to emit light in the visible spectrum, wherein the direction of the emitted light is parallel to the viewing direction of a person wearing the magnification loupe (1);
(b) a spectacle frame (10);
(c) a magnifying optical system (40);
(d) a light source (100) structured and configured to emit an infrared (IR) light beam, wherein said IR light beam is parallel to the viewing direction of a person wearing the magnification loupe (1);
(e) a sensor (110) structured and configured to detect whether the IR light beam generated by the light source (100) is reflected in the direction of the magnification loupe (1); said sensor (110) being structured and configured to activate said light source (60) when the following two conditions are fulfilled:

(i) a reflection of the IR light beam is detected by the sensor (110) and

(ii) the light source (60) is off, said sensor (110) further being structured and configured to deactivate said light source (60) when the following two conditions are fulfilled:

(iii) a reflection of the IR light beam is detected by the sensor (110) and
(iv) the light source (60) is on;
   and
   (f) an energy storage component (90) that is structured and configured to provide electric energy to the light source (60) and that is further structured and configured to provide electric energy to the light source (100).

The spectacle frame (10) typically comprises a right temple (11) and a left temple (12). The magnification loupe (1) of the third aspect may additionally comprise a right spectacle lens (13) and a left spectacle lens (14). The spectacle lenses (13, 14) need not be corrective lenses; they may be simple glass panes. However, for users that need corrective lenses, it is also possible to use such corrective lenses as the right and left spectacle lenses (13, 14) of the magnification loupe (1). The magnifying optical system (40) can be fixed to the right spectacle lens (13) and to the left spectacle lens (14).

Preferably, light source (100) comprises one or more infrared LEDs.

Advantageously, light source (100) and sensor (110) are fixed to the spectacle frame (10) in such a manner that light source (100) and sensor (110) are immediately adjacent to each other so that the sensor (110) can detect IR light beams that are reflected in the direction of light source (100). Preferably, light source (100) and sensor (110) are fixed to the spectacle frame (10) close to or at the nose bridge.

The light source (60) can also be fixed close to the nose bridge but it can also be mounted to the spectacle frame (10) at a position above the right and/or left spectacle lens (13, 14). It is also possible to have two or more light sources (60), e.g. one light source (60) positioned above the right spectacles lens (13) and another light source (60) positioned above the left spectacle lens (14). Alternatively, a light source (60) can also be fixed to a temple (11, 12) of the spectacle frame (10). It is also possible to have two (or more) light sources (60), wherein the first light source (60) is fixed to the right temple (11), and the second light source (60) is fixed to the left temple (12).

The combination of a light source (100) structured and configured to emit an infrared (IR) light beam and a sensor (110) structured and configured to detect whether the IR light beam is reflected in the direction of the magnification loupe (1) allows applications in which the main light source (60) is automatically turned on, when light is needed and automatically turned off, when light is no longer needed. To achieve this effect, the area to be illuminated (e.g. an area of surgical operation; a patient's opened mouth) can be surrounded by a zone that reflects IR light. For example, a ring (200) as further described below can be placed around the area to be illuminated. Alternatively, retroreflective tape can be positioned around the area to be illuminated. At the beginning of an examination or an operation, the user (e.g. a practitioner, a dentist or a surgeon) will turn his gaze from outside the area to be illuminated to the inside of said area to be illuminated. By turning his gaze, the IR light beam emanating from light source (100) crosses the reflective zone and is reflected towards the magnification loupe (1). This reflection of the IR light beam is detected by sensor (110). The main light source (60) is controlled by a circuitry or a similar device in such a manner that the light source (60) is now turned on, because the above conditions (i) and (ii) are fulfilled; i.e. the light source (60) was previously off and the sensor (110) detects a reflection. The light source (60) will remain turned on, as long as the user looks at the area to be illuminated. When the user turns his head to look elsewhere (i.e. away from the area to be illuminated), the IR light beam emanating from light source (100) crosses the reflective zone from the inside to the outside and is reflected towards the magnification loupe (1). This reflection of the IR light beam is detected by sensor (110). The circuitry (or a similar device) controlling light source (60) will turn light source (60) off, since above conditions (iii) and (iv) are fulfilled; i.e. the light source (60) was previously on and the sensor (110) detects a reflection. As long as the user looks away from the area to be illuminated, light source (60) will remain turned off. As soon as the user turns his head to look once again at the area to be illuminated, light source (60) will once again be turned on by the process described above.

The above-described arrangement and process makes sure that light source (60) is only turned on, when really needed. By turning light source (60) off, when it is not needed, the energy consumption of the magnification loupe is reduced, so that the energy storage component (90) will last longer. Thus, the user may wear the magnification loupe (1) for an extended period of time before the magnification loupe (1) has to be connected to a power supply, e.g. to a battery charging station (300).

In preferred embodiments of the first, second or third aspect, the light source (60) which is structured and configured to emit light in the visible spectrum comprises one or more LEDs.

In preferred embodiments of the first, second or third aspect, the magnification loupe further comprises means (120) for cooling the light source (60) which is structured and configured to emit light in the visible spectrum. Preferably, said means (120) for cooling is selected from the group consisting of cooling fins (121) and Peltier elements (122).

In a fourth aspect the present invention is directed to a ring (200) having dimensions that allow the ring (200) to be placed around the opened mouth of a human being, wherein said ring (200) reflects infrared light.

In a preferred embodiment of the fourth aspect, the ring (200) has a surface that is covered with retroreflectors that reflect infrared light. For example, the surface may be covered with corner reflectors, cat's eyes, or retro-reflective tape. The corner reflectors usable in the present invention can be designed similar to the corner reflectors commonly used in bicycle tail lights. The cat's eyes usable in the present invention can be designed similar to cat's eyes that are employed as the retroreflective safety device used in road marking. Retroreflective tape usually comprises a reflective underground into which small glass spheres (typically with a diameter of about 50 μm) are embedded. However, it is essential that the retroreflectors (corner reflectors, cat's eyes or retroreflective tape) used in the present invention reflect infrared (IR) light. Advantageously, the retroreflectors used in the present invention only reflect infrared (IR) light but no light in the visible range.

In another embodiment of the fourth aspect, the cross-section of the ring surface has the form of a paraboloid. It is further preferred that the ring comprises a material that reflects infrared light and/or the ring comprises a coating that reflects infrared light. It is advantageous for practicing the present invention, if the reflecting material and the reflecting coating only reflect infrared (IR) light but no light in the visible range. A cross-section in the form of a paraboloid will make sure that a light beam is reflected in the direction from where it came, irrespective from the original direction of the light beam. Thus, any light beam will be more or less reflected back to the light source producing said light beam.

The ring of the fourth aspect can advantageously be used with the magnification loupe (1) according to the third aspect. More specifically, ring (200) constitutes a zone that reflects IR light, as described above. When using ring (200) in combination with the magnification loupe (1) according to the third aspect, light source (60) is automatically turned on, when light is needed and automatically turned off, when light is no longer needed. The mechanisms through which this effect is obtained are described above.

In a fifth aspect the present invention is directed to a kit-of-parts comprising the magnification loupe (1) according to the third aspect and at least one ring (200) according to the fourth aspect.

In a sixth aspect the present invention is directed to a battery charging station (300) that is formed so that the magnification loupe (1) according to any one of the first, second or third aspect can be placed on said battery charging station (300), wherein said battery charging station (300) is structured and configured to recharge the energy storage component (90) of the magnification loupe (1) by inductive charging when the magnification loupe (1) is placed on said battery charging station (300).

In a seventh aspect the present invention is directed to a kit-of-parts comprising: at least one magnification loupe (1) according to any one of the first, second or third aspect; and a battery charging station (300) according to the sixth aspect.

### LISTING OF REFERENCE NUMERALS

1 magnification loupe
10 spectacle frame
11 right temple
12 left temple
13 right spectacle lens
14 left spectacle lens
40 magnifying optical system
60 light source (structured and configured to emit light in the visible spectrum)
80 energy harvesting component
81 piezoelectric element
82 photovoltaic cell
90 energy storage component
91 rechargeable battery
92 polymer battery
100 light source (structured and configured to emit light in the infrared (IR) spectrum)
110 infrared (IR) sensor
120 means for cooling
121 cooling fins
122 Peltier element
200 ring
300 battery charging station

1. A magnification loupe, comprising:
(a) a light source structured and configured to emit light in the visible spectrum, wherein a direction of the emitted light is parallel to a viewing direction of a person wearing the magnification loupe;
(b) a spectacle frame;
(c) a magnifying optical system;
(d) an energy harvesting component that converts acoustic energy or light energy to electric energy, thereby generating electric energy; and
(e) an energy storage component that is structured and configured to store the electric energy received from the energy harvesting component and that is further structured and configured to provide electric energy to the light source.

2. The magnification loupe of claim 1, wherein said energy harvesting component converts acoustic energy to electric energy and wherein said energy harvesting component is a piezoelectric element.

3. The magnification loupe of claim 1, wherein said energy harvesting component converts light energy to electric energy and wherein said energy harvesting component is a photovoltaic cell.

4. The magnification loupe of claim 1, wherein the energy storage component is a rechargeable battery.

5. A magnification loupe comprising:
(a) a light source structured and configured to emit light in the visible spectrum, wherein a direction of the emitted light is parallel to a viewing direction of a person wearing the magnification loupe;
(b) a spectacle frame, wherein said spectacle frame comprises an energy storage component, said energy storage component being a polymer battery that is structured and configured to provide electric energy to the light source; and
(c) a magnifying optical system.

6. The magnification loupe of claim 5, wherein the spectacle frame comprises two temple portions and wherein at least one of said temple portions is made from a polymer battery.

7. The magnification loupe of claim 6, wherein the polymer battery is a lithium ion polymer battery.

8. The magnification loupe of claim 6, wherein the polymer battery is rechargeable.

9. A magnification loupe, comprising
(a) a first light source structured and configured to emit light in the visible spectrum, wherein a direction of the emitted light is parallel to a viewing direction of a person wearing the magnification loupe;
(b) a spectacle frame;
(c) a magnifying optical system;
(d) a second light source structured and configured to emit an infrared (IR) light beam, wherein said IR light beam is parallel to the viewing direction of the person wearing the magnification loupe;
a sensor structured and configured to detect whether the IR light beam generated by the second light source is reflected in the direction of the magnification loupe;
said sensor being structured and configured to activate said first light source when the following two conditions are fulfilled:
(i) a reflection of the IR light beam is detected by the sensor and
(ii) the first light source is off,
said sensor further being structured and configured to deactivate said first light source when the following two conditions are fulfilled:
(iii) a reflection of the IR light beam is detected by the sensor and
(iv) the first light source is on;
and
(f) an energy storage component that is structured and configured to provide electric energy to the first light source and that is further structured and configured to provide electric energy to the second light source.

10. The magnification loupe according to claim 1, wherein the light source which is structured and configured to emit light in the visible spectrum comprises one or more LEDs.

11. The magnification loupe according to claim 1, further comprising means for cooling the light source which is structured and configured to emit light in the visible spectrum.

12. The magnification loupe according to claim 11, wherein said means for cooling is selected from the group consisting of cooling fins and Peltier elements.

13. A ring having dimensions that allow the ring to be placed around the opened mouth of a human being, wherein said ring reflects infrared light.

14. The ring of claim 13, wherein the ring has a surface that is covered with retroreflectors that reflect infrared light.

15. The ring of claim 13, wherein the cross-section of the ring surface has the form of a paraboloid.

16. The ring of claim 15, wherein the ring comprises a material that reflects infrared light.

17. The ring of claim 15, wherein the ring comprises a coating that reflects infrared light.

18. A kit-of-parts comprising the magnification loupe according to claim 9 and at least one ring having dimensions that allow the rink to be placed around the opened mouth of a human being, wherein said ring reflects infrared light.

19. A battery charging station that is formed so that the magnification loupe according to claim 1 is placed on said battery charging station, wherein said battery charging station is structured and configured to recharge the energy storage component of the magnification loupe by inductive charging when the magnification loupe is placed on said battery charging station.

20. A kit-of-parts comprising:
at least one magnification loupe according to claim 1; and
a battery charging station that is formed so that the magnification loupe is placed on said battery charging station, wherein said battery charging station is structured an configured to recharge the energy storage component of the magnification loupe by inductive charging when the magnification loupe is placed on said battery charging station.

21. The magnification loupe according to claim 9, wherein the first light source which is structured and configured to emit light in the visible spectrum comprises one or more LEDs.

22. The magnification loupe according to claim 21, further comprising means for cooling the first light source which is structured and configured to emit light in the visible spectrum.

23. A battery charging station that is formed so that the magnification loupe according to claim 9 is placed on said battery charging station, wherein said battery charging station is structured and configured to recharge the energy storage component of the magnification loupe by inductive charging when the magnification loupe is placed on said battery charging station.

24. A kit-of-parts comprising:
at least one magnification loupe according to claim 9; and
a battery charging station that is formed so that the magnification loupe is placed on said battery charging station, wherein said battery charging station is structured and configured to recharge the energy storage component of the magnification loupe by inductive charging when the magnification loupe is placed on said battery charging station.
